(19) United States
(12) Patent Application Publication
(10) Pub. No.: US 2002/0081284 A1
(43) Pub. Date: Jun. 27, 2002

(54) Manipulation of arterial-venous identity

(76) Inventor: Dean Y. Li, Salt Lake City, UT (US)

Correspondence Address: J. Matthew Buchanan, BRINKS HOFER GILSON & LIONE, P.O. Box 10395, Chicago, IL, 60610, US

(21) Appl. No.: 09/921,771

(22) Filed: Aug. 3, 2001

Related U.S. Application Data
(60) Provisional application No. 60/222,759, filed on Aug. 3, 2000.

(51) Int. Cl. A61K 48/00; C12N 15/86

(52) U.S. Cl. 424/93.21; 435/456; 514/44

(57) ABSTRACT

Methods and compositions for manipulating the arterial-venous identity of endothelial cells are provided. The methods comprise introducing an arterial molecular program into endothelial cells of a vein section such that the endothelial cells can remodel to form arterial endothelial cells. The arterial molecular program can comprise one or more polynucleotides encoding various genes that are associated with arterial development and/or differentiation from veins. Expression vectors comprising the genes can be used to introduce the molecular program into the cells. A method of treating a patient having an obstructed blood vessel is also provided.

### REFERENCE TO PREVIOUS APPLICATION

This application claims the benefit of U.S. Provisional Application Serial No. 60/222,759 filed on Aug. 3, 2000.

### FEDERALLY SPONSORED RESEARCH OR DEVELOPMENT

The U.S. Government may have rights in the present invention pursuant to the terms of grant number HL65648-01 awarded by the National Institutes of Health and grant number HL03490-01 awarded by the National Heart, Lung and Blood Institute.

### FIELD OF THE INVENTION

The present invention relates to methods and compositions for manipulating the arterial-venous identity of endothelial cells. More particularly, the invention relates to methods of inducing arterial morphology in a vein by transferring an appropriate polynucleotide into endothelial cells of the vein. Further, the invention relates to methods of treating a patient having an obstructed blood vessel.

### BACKGROUND OF THE INVENTION

Obstruction of blood vessels diminishes the ability of the vessel to deliver blood to downstream organs, which impacts the long-term health of the organ and its host. These obstructive disorders are generally referred to as arteriosclerosis. Atherosclerosis, a specific form of arteriosclerosis, primarily affects the aorta and the coronary arteries.

Replacement of the obstructed vessel with a graft of some type is the mainstay of surgical treatment for obstructive vascular disease. A common example of this type of procedure is coronary artery bypass grafting (CABG). This procedure is aimed at alleviating poor blood perfusion of the heart that results from obstructed coronary arteries, and represents the great majority of vessel replacement procedures in humans. In a CABG procedure, a surgeon removes an obstructed segment of the coronary artery and replaces it with a graft.

Choice of graft in any vessel replacement procedure is affected by numerous factors, including rejection by the host immune system, vessel size, and ease of harvesting and handling. For CABG procedures, autologous vessels, particularly the saphenous vein, are predominantly used as the replacement vessel. The saphenous vein, which ascends along the inner side of the leg, is relatively easy to harvest and has a suitable cross-sectional size relative to the coronary arteries. Furthermore, as an autologous vessel, tissue rejection concerns are eliminated. Considering these advantages, it is not surprising that replacement of an obstructed coronary artery section with a section of an autologous saphenous vein has become a common surgical technique for CABG procedures.

Despite these advantages, significant drawbacks remain. For example, recent estimates indicate that as many as 30% of patients who require a CABG procedure do not have veins suitable for grafting. (See Bourassa, Curr. Opin. Cardiol. 9: 685-691 (1994)). Furthermore, approximately 50% of venous bypass grafts are no longer patent, i.e., structurally intact, ten years after grafting, (See Edwards, et al., Surg. Gynecol. & Obstet. 122: 37-42 (1996). The loss of patency of the graft has serious consequences: at a minimum, it can create a need for an additional surgical procedure and, as a worst case, can lead to heart damage and death. Considering these limitations and the many benefits of bypass grafting, there is tremendous interest in improving the patency of vascular grafts.

The use of a vein segment in place of an artery segment probably contributes to the loss of patency in grafts. Arteries have various structural features that are not present in veins. For example, while veins are typically composed of a single layer of endothelium surrounded by a relatively low number of vascular smooth muscle cells, the endothelium of arteries are surrounded by alternating rings of elastic lamellae and vascular smooth muscle cells. These structural differences allow arteries to accommodate different physiological conditions than veins. For example, arteries are typically under higher hemodynamic stress (70-105 mm Hg) than veins (0-8 mm Hg).

Because of these structural features, the use of arteries as grafts has been explored. Indeed, a relatively high percentage of internal mammary artery (IMA) grafts remains intact for years after the grafting procedure. (See Barner, et al., J. Thorac. Cardiovasc. Surg. 90:668-75 (1985)). Thus, the use of arteries appears to have advantages over the use of vascular grafts. Unfortunately, arteries are frequently difficult to harvest. Various technical difficulties are associated with preparation of the IMA and other arteries, such as the gastroepiploic and splenic arteries, placing these vessels in disfavor as replacement grafts.

An ideal graft for replacement of an obstructed section of an artery could be a vessel that combines the benefits of veins, such as ease of harvesting, with those of arteries, such as the above-mentioned structural features.

During embryonic development, endothelial tubes have the capacity to develop into both veins and arteries. The endothelial tubes acquire a specific identity as either an artery or vein prior to the development of the structural features that distinguish the two types of vessels. Molecular programs, comprising various genes and gene products, regulate the identity of these vessels as either arterial or vascular tissue. However, the mere replacement of a vein under arterial hemodynamic conditions does not lead to the transformation of the vein into an artery.

### SUMMARY OF THE INVENTION

The present invention provides methods of inducing arterial morphology in a vein. The method comprises changing the arterial-venous identity of endothelial cells in a segment of a vein to resemble that of endothelial cells in arterial tissue. With an arterial identity, the cells and surrounding tissue can undergo endothelial remodeling such that the vein develops the morphology of an artery, which can improve the ability to serve as vessel replacement grafts.

In a preferred embodiment, the method comprises changing the arterial-venous identity of endothelial cells in vascular tissue by transferring an appropriate polynucleotide(s) into the endothelial cells. The polynucleotide(s) encodes a gene or genes capable of inducing endothelial remodeling of the cells such that the cells resemble endothelial cells associated with an artery. Preferred genes for use in this method include those that function to allow arteries and veins to develop distinct identities, such as endoglin and activin receptor-like kinase I (Alk-1), and those that are differentially expressed in arteries and veins, such as ephrin-B2, Eph B4, elastin and CD34. These genes can be used individually or in any combination. By introducing an appropriate polynucleotide, the endothelial cells of the vascular tissue can remodel and transform their structure to those of an artery.

Furthermore, the present invention also provides methods of treating a patient having an obstructed blood vessel, such as a patient presenting atherosclerosis. In a preferred embodiment, the method comprises harvesting a section of a vein, such as a section of an autologous saphenous vein, changing the arterial-vascular identity of the section by transferring an appropriate polynucleotide into the endothelial cells of the section, removing the obstructed section of a vessel, such as a coronary artery, and grafting the section having the changed arterial-vascular identity for the obstructed section.

### DETAILED DESCRIPTION OF THE PREFERRED EMBODIMENTS OF THE INVENTION

The following description of various preferred embodiments of the invention provides examples of the present invention. The embodiments discussed herein are merely exemplary in nature, and are not intended to limit the scope of the invention in any manner. Rather, the description of these preferred embodiments serves to enable a person or ordinary skill in the relevant art to practice the present invention.

In one embodiment, the present invention provides methods and compositions for manipulating the arterial-venous identity of endothelial cells. Manipulation of the arterial-venous identity is accomplished by transferring one or more polynucleotides, or products thereof, that encode one or more genes capable of inducing remodeling of the cells such that the cells resemble endothelial cells associated with an artery. The polynucleotide encodes genes that belong to one or both of the following classes:

1 ) genes that have a function of allowing endothelial cells to develop distinct artery or vein identities; and

2) genes that are differentially expressed in endothelial cells of arteries and veins.

The development of arterial-venous identity is one step in the pathway that allows embryonic endothelial tubes to develop into either of these types of vessels. Furthermore, the inventor has discovered that insertion of the polynucleotide described above into venous endothelial cells allows the cells to remodel into arterial endothelial cells. The development of an arterial identity is measured by the appearance of arterial structural features.

The first class of genes that can be used comprises those genes that have a function of allowing endothelial cells, during embryonic development, to develop distinct identities as being either arterial or venous. During development, vascular remodeling and endothelial maturation produce the final vasculature system. To form an organized vascular network, a hierarchy of major and minor vessels that efficiently transport blood to and from tissues must be established. The formation of a mature hierarchical vascular system forms in two steps. The first step involves the differentiation, rapid proliferation and tube formation of endothelial cells. This process results in the formation of a meshwork of interconnected and homogeneously sized endothelial tubes. In the second stage, vascular remodeling and endothelial maturation occurs. Endothelial tubes must be distinguished as arterial or venous, and an organized network is formed through differential growth, apoptosis, and sprouting of endothelial tubes. This process of remodeling leads to a well-defined vascular network that efficiently supplies blood to and removes waste from the target tissue or tumor. Endoglin and activin receptor-like kinase 1 (Alk1) function to initiate the switch from stage I-endothelial differentiation and rapid proliferation and stage 2-endothelial maturation and vascular remodeling. (See generally, Ferrara N, and Alitalo, K. Nat. Med. 5:1359-64 (1999), Folkman J., and D¹Amore P. A., Cell 87:1153-1155 (1996), Gale N. W., and Yancopoulos G. D., Genes Dev 13(9): 1055-66 (1999), Inducing Alk-1 and endoglin function will promote maturation and the molecular program needed to distinguish arteries from veins. Disrupting the function of these genes will prevent a nascent endothelial network from forming a highly organized network. Thus, blocking the function of these genes may prevent pathologic neovascularization, processes that are critical for cancer growth and diabetic retinopathy. See, for examples, Li, D. Y., et al., Science 284:1534-1537 (1999) and Urness, L. D., et al., Nature Genetics 26:328-331 (2000). Examples of genes belonging to this first class include endoglin and activin receptor-like kinase 1 (Alk-1).

Endoglin is a transforming growth factor-β (TGF-β) binding protein expressed on the surface of endothelial cells. TGF-β signaling is required for vasculogenesis, the first stage of vascular development. During vasculogenesis, the primary capillary network, composed of interconnected and homogenously sized endothelial tubes, is formed. Indeed, mice lacking endoglin die at an early age due to defective vascular development characterized by poor smooth muscle development and arrested endothelial remodeling. Consequently, endoglin is essential for the second stage of vascular development, angiogenesis, in which the primary endothelial network is remodeled into a mature circulatory system. See generally Li, D. Y., et al., Science 284:1534-1537 (1999).

The cDNA encoding endoglin has been described. (Gougos, A. and Letarte, M., J. Biol. Chem. 265(15): 8361-8364 (1990)). The sequence is appended hereto as SEQ ID NO. 1.

The Alk-1 gene encodes a serine/threonine kinase receptor for the TGF-β superfamily of growth factors (ten, Dijke, P., et al, Science 264 (5155): 101-4 (1994); ten, Dijke, P., et al., Oncogene 10: 2879-87 (1993); Attisano, L. & Wrana, J. L., Cytokines and Growth Factor Reviews 7(4): 327-339 (1996)). The receptor encoded by Alk-I is highly expressed in the endothelium (Roelen, B. A., et al, Dev. Dyn. 209(4): 418-30 (1997)). Also, loss-of-function mutations of Alk-I are responsible for a human vascular dysplasia characterized by arteriovenous malformations (Guttmacher, A. E., et al., N. Engl. J. Med. 333(14): 918-924 (1995); Johnson, D. W., et al., Nat. Genet. 13(2): 189-95 (1996)). Furthermore, anatomical, molecular, and functional distinctions between arteries and veins are lost in mice lacking Alk-1. Lastly, Alk-1 is required for successful embryonic development of distinct arterial and venous vascular beds (id.).

The cDNA encoding Alk-1 has been described (ten Dijke, P. P., et al., Oncogene 8(10): 2879-2887 (1993)). The sequence appended hereto as SEQ ID NO. 2

The second class of genes that can be used in the arterial molecular program comprises those genes that are differentially expressed in the endothelial cells of arteries and veins. As used herein, the term “differentially expressed” refers to the relative extent of expression of a gene in an endothelial cell in an artery as compared to an endothelial cell in a vein. Examples of genes belonging to this second class include ephrin-B2, EphB4, elastin, and CD34. See, for example, Urness, L. D., et al., Nature Genetics 26:328-331 (2000).

The ephrin-B2 gene encodes an arterial specific molecular marker that is expressed prior to the appearance of any structural or functional differences between arteries and veins (Adams, R. H., et al. Gened Dev. 13:3 295-306 (1999), Wang, H. U., et al., Cell 93(5): 741-53 (1998)). Also, while mice lacking the ephrin-B2 gene or the gene for the ephrin-B2 receptor, EphB4, develop distinct arterial and venous domains, these mice experience defective endothelial remodeling (Id.; Gerety, S. S., et al., Mol Cell 4:403-14 (1999)). Thus, while ephrin-B2 and EphB4 are important arterial markers, they do not regulate the specification of endothelial tubes to become arteries and veins. Indeed, mice lacking the Alk-1 gene fail to express normal levels of these markers despite the presence of an extensive endothelial network. (Urness, L. D., et al., Nature Genetics 26: 328-331 (2000)).

The cDNA encoding ephrin-B2 has been described. (Bennett, B. D., et al., Proc. Natl. Acad. Sci. U.S.A. 92(6): 1866-1870 (1995)). The sequence is appended hereto as SEQ ID NO. 3

The cDNA encoding EphB4 appended hereto as SEQ ID NO. 4

Elastin is the main component of the extracellular matrix of arteries. Elastin has both structural and developmental roles. During arterial development, elastin controls proliferation of smooth muscle and stabilizes arterial structure. Indeed, mice lacking elastin die of an obstructive arterial disease resulting from subendothelial cell proliferation and reorganization of smooth muscle. (See Li, D. Y., et al., Nature 393:276-280 (1998)).

The cDNA for elastin has been described (Faszio, M. J., et al., J. Invest. Dermatol. 91(5) 458-464 (1998). The sequence is appended hereto as SEQ ID NO. 5.

The CD34 gene encodes a cell surface glycoprotein that is expressed in early blood vessels, as well as on various hematopoietic cells (See, Wood, H. B., et al., Blood 90(6): 2300-2311 (1977)).

The cDNA encoding CD34 has been described (NCBI Annotation Project, Direct Submission, 7-16-2001). The sequence is appended hereto as SEQ ID NO. 6

Preferably, transferring the polynucleotide(s) into an endothelial cell having a venous identity is accomplished by transferring an expression vector comprising one or more of the genes described above. Suitable expression vectors useful in accordance with the present invention include eukaryotic, plasmid and viral vectors, and combinations thereof. Examples of useful viral vectors include recombinant viral vectors such as adenoviral, retroviral, herpesviral, pox viral, and adeno-associated viral vectors. Preferably, the polynucleotides are contained within the expression vector. Also preferable, the expression vector is adapted to introduce the polynucleotide into the endothelial cells.

The transferring of the polynucleotide into the endothelial cells can occur in vivo or ex vivo. Preferably, the transferring occurs ex vivo on a vessel segment harvested from a patient. Conventional transduction techniques can be utilized to carry out the ex vivo transferring of the polynucleotide into the endothelial cells when viral vectors are used. Examples of suitable transduction techniques include those described in Kibbe, M. R., et al., J. Vasc. Surg.34(1): 156-65 (2001) and Moawad, J., et al., Ann. Vasc. Surg. 15(3):367-73 (2001). The transduction should be carried out using a sufficient number of vector particles to ensure adequate transferring of the polynucleotide. Also, the transduction should be carried out under culturing conditions that are conducive to the viability of the endothelial cells as well as the transduction by the vector. Preferred number of vector particles and length of transduction period for changing the arterial venous identity of a segment of a saphenous vein are between approximately 1×10⁸ and 1×10¹² viral particles for 15 to 45 minutes. Particularly preferable, approximately 1×10¹⁰ to 1×10¹² viral particles are exposed to the vein segment for approximately 30 minutes. Most preferable, approximately 1×10¹¹ viral particles are exposed to the endothelial cells of the vein segment for approximately 30 minutes.

The genes may be encoded on a plasmid or other similar construct and then incorporated into the vector. Conventional molecular biology techniques can be employed to create suitable constructs for use in the present invention.

Preferred viral vectors include recombinant retroviral and adeno-associated viral vectors. Recombinant retroviral vectors are frequently used for gene transfer, and methods for constructing such vectors are known in the art (Hodgson, Bio/Technology 13: 222-225 (1995); Miyanohara, et al., Proc. Natl. Acad. Sci. USA 85: 6538-6542 (1988); Rosenberg, et al., New Engl. J. Med. 323: 570-578 (1990)). Preferably, retroviral vectors with impaired ability to replicate and transform are used.

Methods for producing recombinant adeno-associated viral (AAV) vectors are also known in the art. Briefly, a suitable producer cell line is transfected with an AAV vector containing the gene of interest, which can be encoded on a plasmid. AAV helper functions (i.e., the products of the AAV rep and cap genes) and accessory functions, which are typically derived from a helper virus, such as adenovirus or herpesvirus, are then expressed in the producer cell. Once these factors come together, the gene(s) of interest is (are) replicated and packaged as though it were a wild-type AAV genome, forming a recombinant virion. When cells, such as endothelial cells, are infected with the resulting AAV virions, the gene(s) of interest enter the cell and is (are) expressed. Because the cells lack the rep and cap genes and the helper virus accessory function genes, the rAAV are replication defective; that is, they cannot further replicate and package their genomes. Similarly, without a source of rep and cap genes, wild-type AAV cannot be formed in the infected cell. For a detailed discussion on the production of rAAV virions, see U.S. Pat. No. 6,001,650 to Colosi for HIGH-EFFICIENCY WILD-TYPE-FREE AAV HELPER FUNCTIONS.

The polynucleotides encoding the gene(s) of interest can be inserted into the expression vectors and used for cell transfection using conventional recombinant techniques, such as those described by Sambrook, Fritsch & Maniatis, in “Molecular Cloning, A Laboratory Manual” (2d ed): pp. E.5 (Cold Spring Harbor Press, Cold Spring Harbor, N.Y. 1989). Alternatively, the expression vectors can be prepared using homologous recombination techniques, such as those described by Davidson, et al., Nature Gen. 3: 219-223. (1993) and Lemarchand, Proc. Natl. Acad. Sci. USA 89(14): 6482-6486 (1992).

The expression vectors of the present invention can additionally contain regulatory elements such as promoters, as well as selection markers, such as antibiotic resistance genes. Furthermore, the expression vectors can include tags that allow for binding of the protein of interest to a binding agent of some sort, which can be used to facilitate purification and/or localization and targeting efforts. Various such tags are known to those skilled in the art. Examples include F_{c} receptors and Hexo-histidine tags.

It is well established that viral vectors will be taken up into and integrated into cells in vivo, to eventually express the viral DNA₇ including any inserted constructs (Nabel, U.S. Pat. No. 5,328,470; Yoshimura, et al., J. Biol. Chem. 268(4): 2300-2303 (1993); Crystal, AM. J. Med. 92(6A): 445-525 (1992); Lemarchand, et al., Proc. Natl. Acad. Sci. USA 89(14): 6482-6486 (1992)).

Alternatively, non-viral methods can be used to introduce the polynucleotides into the endothelial cells. Essentially, any suitable method for introducing DNA into cells for later expression can be utilized. For example, techniques such as calcium phosphate co-precipitation (Graham, et al., Virol. 52: 456-467 (1973)), direct micro-injection of DNA into cells (Capecchi, Cell 22: 479-488 (1980)), lipisome-mediated gene transfer (Mannino, et al., BioTechniques 6: 682-690 (1988), lipid-mediated transfection (Feigner, et al., Proc. Natl. Acad. Sci. USA 84: 7413-7417 (1987)), delivery using DNA-coated stents placed at a target site by a catheter, and nucleic acid delivery using high-velocity microprojectiles (Klein, et al., Nature 327: 70-73 (1987)) can be used. Furthermore, electroporation methods for introducing DNA into cells and tissues can be used (Shigekawa, et al., BioTechniques6: 742-751 (1988)).

Those skilled in the art will readily recognize that the various genes introduced into endothelial cells, using either viral or non-viral methods, may be operably linked to control elements such as promoters and enhancers, that are capable of driving or repressing gene expression under appropriate conditions. Termination signals, such as polyadenylation sites, can also be included. Control elements, such as inducible promoters, that allow controlled expression of the gene of interest are available. For example, an ecdysone-inducible promoter can be utilized to regulate gene expression. (See, e.g., Stratagenes; Complete Control™ Inducible Mammalian Expression System Instruction Manual—available online at htt;//ww.stratagene.com/manuals/index.shtm). Other examples of suitable inducible promoters that are functional in mammalian cells include those that are induced (or repressed) by tetracycline and its derivatives, RU486, and rapamycin and its derivatives (See, e.g. Grossen & Brujard, Proc. Natl. Acad. Sci, USA 89: 5547-5551 (1992); Wang, et al., Gene Therapy, 4: 432-441 (1997); and Riviera, et al., Nature Medicine 2:1028-1032 (1996).

The manipulation of the arterial-vascular identity of endothelial cells may also be accomplished by introducing the products of one or more of the above-mentioned genes into the endothelial cells, The gene products may be produced using standard recombinant techniques known to those skilled in the art (See, generally, Sambrook, et al., (supra.)). Recombinantly produced gene products may be purified using conventional purification schemes, such as affinity chromatography, size-exclusion, filtration, precipitation, and other suitable techniques. The gene products can be introduced into the endothelial cells using techniques such as microinjection and protein transduction (see, e.g., Schwarze, et al, Science 285: 1569-1572 (1999)).

The present invention also provides a composition comprising a blood vessel, such as a section of a vein or a vascular graft, having endothelial cells comprising an exogenously supplied polynucleotide encoding a gene that is capable of inducing endothelial remodeling. The gene can be any of the genes described above in the description of the methods of the present invention. Thus, the gene can comprise endoglin, Alk-1, ephrin-B2, EphE4, elastin, and/or CD34. The compositions of the present invention are useful as grafts to replace sections of arteries containing obstructions, such as coronary arteries affected by atherosclerosis.

In one embodiment, the composition of the present invention comprises a section of an autologous vein, i.e., a section of a vein of the patient ultimately receiving the graft. The use of autologous tissue eliminates any tissue rejection concerns.

Any suitable vein can be utilized for the vein section. The choice of vein will depend on various factors, such as ease of harvesting, ability of the vein to tolerate removal of a section, and the relative capacity of the vessel as compared to that of the obstructed vessel. Preferred veins include the saphenous vein of the leg. Particularly,preferable, the vein section comprises a section of the internal or long saphenous vein. Sections of these preferred veins can be readily harvested by surgical techniques known to those skilled in the art.

Importantly, the vein section must include the endothelial cell layer (endothelium) such that the polynucleotide and gene can be introduced into the endothelial cells. The polynucleotide can encode any combination of the genes discussed above. Also, the polynucleotide can be introduced into the endothelial cells of the vein section according to any suitable technique, such as those described above.

Alternatively, the composition can comprise an engineered blood vessel comprising endothelial cells.

Engineered blood vessels are vessels fabricated from tissue engineering procedures. This class of vessel includes synthetic material in combination with natural cells, such as endothelial cells, as well as cultured vessels produced from natural materials, such as smooth muscle and endothelial cells. Examples of such vessels, as well as techniques for their production, can be found in Huynh, T., et al., Nature Biotechnology, 17: 1083-1086 (1999); Niklason, L. E., et al., Science 284: 489-493 (1999); L'Heureux, et al, FASEB J. 12: 47-56 (1998); and Campbell, J. H., et al., Cir. Res. 85: 1173-1178 (1999).

The expression vectors of the present invention can be introduced into endothelial cells of an engineered blood vessel in the same manner as that described above for segments of natural veins.

The present invention also provides a method of treating a patient having an obstructed blood vessel. The method of treatment can be practiced on any mammal, but is particularly well-suited for treating humans. The method is particularly well-suited for treating patients having obstructed arteries, such as coronary arteries affected by atherosclerosis.

As detailed above, surgical grafting of a vascular graft in place of the obstructed artery is a common surgical technique for treating patients with obstructed vessels. The method of the present invention can be practiced in accordance with guidelines known in the art, such as those relating to the need for bypass grafting as a function of the fraction of the vessel blocked.

In a preferred embodiment, the method of treatment comprises providing a graft comprising endothelial cells, changing the arterial-vascular identity of the endothelial cells by transferring a polynucleotide encoding a gene capable of inducing endothelial remodeling into the endothelial cells, removing an obstructed section of a vessel of the patient, and grafting the graft having the endothelial cells with changed arterial-vascular identity in place of the removed obstructed section. The arterial-vascular identity of the endothelial cells can be changed ex vivo prior to grafting, or in vivo after grafting.

The graft can comprise a vein section or an engineered blood vessel, as described above. If the graft comprises a vein section, the section preferably comprises a section of an autologous vein, and particularly preferably comprises a section of a saphenous vein of the patient. Also, if the graft comprises a vein section, the method may further comprise harvesting the vein section from a vein of the patient. The harvesting can be accomplished according to conventional techniques known in the art.

The changing the arterial-vascular identity of the endothelial cells by transferring an appropriate polynucleotide into the endothelial cells can be accomplished according to the methods of the present invention, detailed above.

The removing an obstructed section of a vessel of a patient and the grafting of the graft in place of the removed obstructed section can both be accomplished according to conventional techniques known in the art.

### EXAMPLE

The present invention can be carried out to alter the arterial-venous identity of endothelial cells in a vein segment in an ex vivo environment. This preferred method is particularly well-suited for treating a segment of a vein that has been harvested from a patient suffering from an obstructed blood vessel. The treated vein segment can be used as a graft to replace an obstructed section of the obstructed vessel.

When practicing this method, a segment of a saphenous vein of the patient will be harvested according to conventional surgical procedures. The section will be dissected to provide a segment that is of a suitable length, i.e. a length sufficient to allow the segment to serve as a replacement for the obstructed section of the obstructed vessel.

The vein segment will be passively transduced with an adenoviral vector carrying one or more genes encoding Alk-1, endoglin, ephrin-B2, Eph-B4, elastin, and CD34. The transduction will be carried out using approximately 1×10¹¹ adenoviral vector particles for 30 minutes using standard techniques. Examples of suitable transduction techniques are described in Kibbe, M. R., et al., J. Vasc. Surg. 34(1): 156-65 (2001) and Moawad, J., et al., Ann. Vasc. Surg. 15(3):367-73 (2001).

The obstructed section of the obstructed vessel will be removed using conventional surgical techniques. Lastly, the transduced vein segment will be interposed as a graft in place of the obstructed section. Thus, if a coronary artery was the obstructed vessel, the transduced vein segment will be grafted into the coronary circulation in place of the obstructed section. The grafting can occur in the peripheral circulation, if needed, based on the location of the obstructed section of the obstructed vessel.

All references cited and otherwise referred to herein are hereby incorporated in their entirety, except to the extent to which they may contradict any definition or statement herein.

The foregoing disclosure is the best mode devised by the inventor for practicing the invention. It is apparent, however, that several variations in accordance with the present invention may be conceivable to one of ordinary skill in the relevant art. Inasmuch as the foregoing disclosure is intended to enable such person to practice the instant invention, it should not be construed to be limited thereby, but should be construed to include such aforementioned variations. As such, the present invention should be limited only by the spirit and scope of the following claims.

1. A method for inducing arterial morphology in a vein, comprising:
contacting endothelial cells in said vein to at least one polynucleotide encoding a gene that is capable of inducing endothelial remodeling for a time sufficient to transfer the polynucleotide into the endothelial cells.

2. The method of claim 1, wherein said vein is a mammalian vein.

3. The method of claim 1, wherein the vein is a human vein.

4. The method of claim 3, wherein the vein is a saphenous vein.

5. The method of claim 1, wherein the gene encodes endoglin, Alk-1 or both.

6. The method of claim 1, wherein the gene encodes one or more of ephrin-B2, EphB4, elastin and CD34.

7. The method of claim 1, wherein the polynucleotide is contained within an expression vector adapted to introduce the polynucleotide into the cells.

8. The method of claim 7, wherein the expression vector is a viral vector.

9. The method of claim 8, wherein the viral vector is an adenoviral vector, a herpesviral vector, a pox viral vector, or an adeno-associated viral vector.

10. A method of treating a patient having an obstructed blood vessel, comprising:
providing a graft comprising endothelial cells;
contacting the endothelial cells of the graft to at least one polynucleotide encoding a gene that is capable of inducing endothelial remodeling for a time sufficient to transfer the polynucleotide into the endothelial cells;
removing a section of said obstructed blood vessel; and
grafting the graft in place of the removed section of said obstructed blood vessel.

11. The method of claim 10, wherein providing a graft comprises harvesting a section of a vein from said patient.

12. The method of claim 11, wherein the vein is a saphenous vein of said patient.

13. The method of claim 10, wherein the gene encodes endoglin, Alk-1, or both.

14. The method of claim 10, wherein the gene encodes one or more of ephrin-B2, EphB4, elastin and CD34.

15. The method of claim 10, wherein the polynucleotide is contained within an expression vector adapted to introduce the polynucleotide into the cells.

16. The method of claim 15, wherein the expression vector is a viral vector.

17. The method of claim 16, wherein the viral vector is an adenoviral vector, a retroviral vector, a herpesviral vector, a pox viral vector, or an adeno-associated viral vector.

18. A blood vessel, comprising endothelial cells comprising an exogenously supplied polynucleotide encoding a gene that is capable of inducing endothelial remodeling in the endothelial cells.

19. A blood vessel in accordance with claim 18, wherein the vessel is a section of a mammalian vein.

20. A blood vessel in accordance with claim 19, wherein the vessel is a section of a human vein.

21. A blood vessel in accordance with claim 20, wherein the vessel is a section of a saphenous vein.

22. A blood vessel in accordance with claim 18, wherein the gene encodes endoglin, Alk-1 or both.

23. A blood vessel in accordance with claim 18, wherein the gene encodes one or more of ephrin-B2, EphB4, elastin, and CD34.


SEQUENCE LISTING (6 sequences)

SEQ ID NO: 1 - DNA, 3142 bp, Homo sapiens
cctgggccggccgggctggatgagccgggagctccctgctgccggtcataccacagccttcatctgcgccctggggccaggactgctgctgtcactgccatccattggagcccagcaccccctccccgcccatccttcggacagcaactccagcccagccccgcgtccctgtgtccacttctcctgacccctcggccgccaccccagaaggctggagcagggacgccgtcgctccggccgcctgctcccctcgggtccccgtgcgagcccacgccggccccggtgcccgcccgcagccctgccactggacacaggataaggcccagcgcacaggcccccacgtggacagcatggaccgcggcacgctccctctggctgttgccctgctgctggccagctgcagcctcagccccacaagtcttgcagaaacagtccattgtgaccttcagcctgtgggccccgagaggggcgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcggaaaaaggtgcactgcctcaacatggacagcctctctttccagctgggcctctacctcagcccacacttcctccaggcctccaacaccatcgagccggggcagcagagctttgtgcaggtcagagtgtccccatccgtctccgagttcctgctccagttagacagctgccacctggacttggggcctgagggaggcaccgtggaactcatccagggccgggcggccaagggcaactgtgtgagcctgctgtccccaagccccgagggtgacccgcgcttcagcttcctcctccacttctacacagtacccatacccaaaaccggcaccctcagctgcacggtagccctgcgtcccaagaccgggtctcaagaccaggaagtccataggactgtcttcatgcgcttgaacatcatcagccctgacctgtctggttgcacaagcaaaggcctcgtcctgcccgccgtgctgggcatcacctttggtgccttcctcatcggggccctgctcactgctgcactctggtacatctactcgcacacgcgtgagtaccccaggcccccacagtgagcatgccgggcccctccatccacccgggggagcccagtgaagcctctgagggattgaggggccctggcaggaccctgacctccgcccctgcccccgctcccgctcccaggttcccccagcaagcgggagcccgtggtggcggtggctgccccggcctcctcggagagcagcagcaccaaccacagcatcgggagcacccagagcaccccctgctccaccagcagcatggcatagccccggccccccgcgctcgcccagcaggagagactgagcagccgccagctgggagcactggtgtgaactcaccctgggagccagtcctccactcgacccagaatggagcctgctctccgcgcctacccttcccgcctccctctcagaggcctgctgccagtgcagccactggcttggaacaccttggggtccctccaccccacagaaccttcaacccagtgggtctgggatatggctgcccaggagacagaccacttgccacgctgttgtaaaaacccaagtccctgtcatttgaacctggatccagcactggtgaactgagctgggcaggaagggagaacttgaaacagattcaggccagcccagccaggccaacagcacctccccgctgggaagagaagagggcccagcccagagccacctggatctatccctgcggcctccacacctgaacttgcctaactaactggcaggggagacaggagcctagcggagcccagcctgggagcccagagggtggcaagaacagtgggcgttgggagcctagctcctgccacatggagccccctctgccggtcgggcagccagcagagggggagtagccaagctgcttgtcctgggcctgcccctgtgtattcaccaccaataaatcagaccatgaaacctgaaaaaaaaaaaaa

SEQ ID NO: 2 - DNA, 1970 bp, Homo sapiens
aggaaacggtttattaggagggagtggtggagctgggccaggcaggaagacgctggaataagaaacatttttgctccagcccccatcccagtcccgggaggctgccgcgccagctgcgccgagcgagcccctccccggctccagcccggtccggggccgcgccggaccccagcccgccgtccagcgctggcggtgcaactgcggccgcgcggtggaggggaggtggccccggtccgccgaaggctagcgccccgccacccgcagagcgggcccagagggaccatgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacacgatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtattgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaatagcccaggagcacctgattcctttctgcctgcagggggctgggggggtggggggcagtggatggtgccctatctgggtagaggtagtgtgagtgtggtgtgtgctggggatgggcagctgcgcctgcctgctcggcccccagcccacccagccaaaaatacagctgggctgaaacctg

SEQ ID NO: 3 - DNA, 2902 bp, Homo sapiens
cacagccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggagcctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctaga

SEQ ID NO: 4 - DNA, 3945 bp, Homo sapiens
cgtccacccgcccagggagagtcagacctgggggggcgagggccccccaaactcagttcggatcctacccgagtgaggcggcgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggt

SEQ ID NO: 5 - DNA, 2274 bp, Homo sapiens
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcaggtgcagatgagggagttaggcggagcctgtcccctgagctcagggaaggagatccctcctcctctcagcacctccccagcaccccctcatcacccagggtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 6 - DNA, 2615 bp, Homo sapiens
ccttttttggcctcgacggcggcaacccagcctccctcctaacgccctccgcctttgggaccaaccaggggagctcaagttagtagcagccaaggagaggcgctgccttgccaagactaaaaagggaggggagaagagaggaaaaaagcaagaatcccccacccctctcccgggcggagggggcgggaagagcgcgtcctggccaagccgagtagtgtcttccactcggtgcgtctctctaggagccgcgcgggaaggatgctggtccgcaggggcgcgcgcgcagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggaggggcaggtaaactcctgtcctttacacattcggctccctggagccagactctggtcttctttgggtaaacgtgtgacgggggaaagccaaggtctggagaagctcccaggaacaactgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcttttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgaacagggatcctggtttcaatgacggttggaaatagaaatttccagagaagagagtattgggtagatattttttctgaatacaaagtgatgtgtttaaatactgcaattaaagtgatactgaaacac